(19) United States
(12) Patent Application Publication
(10) Pub. No.: US 2018/0015119 A1
(43) Pub. Date: Jan. 18, 2018

(54) PROCESS FOR GRANULATING SEVELAMER CARBONATE

(71) Applicant: Amneal Pharmaceuticals Company GmbH, Steinhausen (CH)

(72) Inventors: Niraj Kumar, Ahmedabad (IN); Santnu Patel, Ahmedabad (IN); Jatin Gajjar, Ahmedabad (IN); Ketan B. Ramani, Ahmedabad (IN)

(73) Assignee: Amneal Pharmaceuticals Company GmbH, Steinhausen (CH)

(21) Appl. No.: 15/548,616

(22) PCT Filed: Feb. 19, 2016
(86) PCT No.: PCT/EP2016/053601

§ 371 Date: Aug. 3, 2017

(30) Foreign Application Priority Data
Feb. 23, 2015 (IN) 590/MUM/2015

(51) Int. Cl. A61K 31/785; A61K 9/20

(52) U.S. Cl. CPC A61K 31/785; A61K 9/2054; A61K 9/2072

(57) ABSTRACT

The present disclosure provides a pharmaceutical composition comprising of a polyallylamine polymer or pharmaceutically acceptable salts thereof; excipient based hydrous granules and optionally moisture retaining agents. The present disclosure also provides process embodiments for preparation of the pharmaceutical compositions comprising of modified moisture activated granulation technique. Polyallylamine polymers used are Sevelamer, Colesevelam or pharmaceutically acceptable salts thereof, preferably Sevelamer HCl, Sevelamer carbonate or Colesevelam HCl. The Tablet according to the present disclosure comprising of polyallylamine polymer which can be prepared by using modified moisture activated granulation technique, by adding one or more excipient based hydrous granule(s), contacting the said hydrous granule(s) with polyallylamine polymer and optionally adding one or more moisture retaining agents and lubricant(s) resulting in the compression into a tablet form using suitable tools.

### FIELD OF THE INVENTION

The present disclosure relates to a pharmaceutical composition constituting of a polyallylamine polymer as an active pharmaceutical ingredient (API) and an excipient based hydrous granule(s) in a tablet form. The disclosure also relates to processes for preparation of such compositions. Embodiments may include a tablet consisting of excipient based hydrous granule(s) and/or moisture retaining agent combined with the API.

### BACKGROUND OF THE INVENTION

Sevelamer and Colesevelam are polyallylamine polymer that are useful as medicinal agent which are disclosed in the prior art literature.

Sevelamer is disclosed in the U.S. Pat. No. 5,667,775. Sevelamer has been approved by the FDA as an active pharmaceutical ingredient and has a salt form in two products viz. Renvela® and Renagel®. Renvela® is a film coated tablet consisting of Sevelamer carbonate as active pharmaceutical ingredient. Renagel® is a tablet consisting of Sevelamer hydrochloride as an active pharmaceutical ingredient which is also available in a powdered form in a sachet for suspension under the same brand name.

Colesevelam is disclosed in the U.S. Pat. No. 5,693,675. Colesevelam has been approved by the FDA as an active pharmaceutical ingredient. It has been approved as a hydrochloride salt form under the Welchol® brand name. Welchol® is a film coated tablet consisting of Colesevelam hydrochloride as an active pharmaceutical ingredient which is also available in a powdered form in a sachet for suspension under the same brand name.

U.S. Pat. No. 6,733,780 discloses the composition of Sevelamer hydrochloride prepared using Direct Compression Technique.

U.S. Pat. No. 7,846,425 discloses the composition of Sevelamer hydrochloride prepared by wet granulation using non-aqueous solvents.

U.S. Pat. No. 7,749,536 discloses the composition of Sevelamer carbonate/hydrochloride prepared by wet granulation using organic solvent/water mixture as the granulating fluid.

US 2011/0189121 discloses slug comprising a polyallylamine polymer technique and method of its preparation by compaction.

Polyallylamine polymer or salts thereof like Sevelamer hydrochloride, Sevelamer carbonate or Colesevelam hydrochloride are poorly compressible and hence difficult to densen into a tablet form. It is previously known that the processes employed to achieve compression into a tablet are either by direct compression, wet granulation or slug compaction technique. All these approaches are either incapable of preparing the tablet with an acceptable hardness, friability and/or disintegration time or involve special instruments and time consuming processes that add to the overall cost of the process. Direct compression of a mixture of polyallylamine polymer and excipients results in amore friable by-product. Furthermore, due to the non-compressible nature and higher concentration of the polyallylamine polymer that is required per tablet, causes difficulty in compressibility.

There is a growing need for alternate ways to formulate compositions of polyallylamine polymers in a tablet form

### SUMMARY OF THE INVENTION

Aspects of the present disclosure relates to pharmaceutical compositions comprising of polyallylamine polymer and salts thereof.

One or more embodiments may include a pharmaceutical composition comprising of polyallylamine polymer and salts thereof manufactured by utilizing the modified moisture activated granulation process. The polyallylamine polymer and salts thereof is either a Sevelamer hydrochloride, a Sevelamer carbonate or a Colesevelam hydrochloride.

In another embodiment, the polyallylamine polymer maybe Sevelamer, Colesevelam or pharmaceutically acceptable salts thereof.

One or more embodiments may include the pharmaceutically acceptable salts of the polyallylamine polymer selected from the group of Sevelamer carbonate, Sevelamer hydrochloride and Colesevelam hydrochloride.

Another aspect of the present disclosure relates to the processes for the preparation of the pharmaceutical compositions comprising of polyallylamine polymer or salts thereof.

The pharmaceutical tablet composition of the present disclosure can be further packaged in to the suitable packaging material for long term storage.

Another aspect of the present disclosure relates to the pharmaceutical tablet compositions comprising of polyallylamine polymer or salts thereof that have an acceptable hardness, friability and/or disintegration time throughout the shelf life period upon storage.

In another embodiment of the disclosure, we have found that using modified moisture activated granulation techniques solves the problem of poor compression i.e. easy tabletting as opposed to prior art tabletting techniques. The tablets that result from modified moisture activated granulation technique have an acceptable hardness and disintegration time. The present disclosure does not involve a drying step and significant agglomeration, making the process simple, effective and cost-efficient.

### DETAILED DESCRIPTION

Aspects of the present disclosure relates to pharmaceutical compositions comprising of polyallylamine polymer or salts thereof.

Examples of polyallylamine polymer disclosed in the prior art literature includes, but are not limited to Sevelamer, Colesevelam or pharmaceutically acceptable salts thereof. Pharmaceutically acceptable salts forms of Sevelamer and Colesevelam disclosed in the prior art literature includes, but are not limited to Sevelamer HCl, Sevelamer carbonate or Colesevelam HCl. The polyallylamine polymer may be present in an amount ranging from about 50% to about 95% by weight of the composition, more preferably ranging from about 60% to about 85% by weight of the composition, most preferably ranging from about 70% to about 80% by weight of the composition.

In one or more preferred embodiments, Sevelamer HCl, Sevelamer carbonate or Colesevelam HCl according to the present disclosure has a moisture content of not more than 11% w/w and more preferably between 5-9% w/w and most preferably around about 7% w/w. In accordance with one aspect, the present disclosure provides a pharmaceutical composition comprising of a polyallylamine polymer or salts thereof and one or more pharmaceutically acceptable excipients. The pharmaceutical composition according to the present disclosure has a moisture content of not more than 17% w/w, preferably between about 8% w/w to about 15% w/w of the said pharmaceutical composition.

The pharmaceutical compositions comprising of polyallylamine polymer or salts thereof and excipient based hydrous granules.

The suitable excipients for the preparation of excipient based hydrous granules, include excipients capable of absorbing moisture.

The excipient based hydrous granules has a moisture content of between 10-35% w/w, or between 15-30% w/w, or between 20-25% w/w or around about 23% w/w. The excipient based hydrous granules may be present in an amount ranging from about 5% to about 30% by weight of the composition, more preferably ranging from about 8% to about 20% by weight of the composition, most preferably ranging from about 10% to about 15% by weight of the composition.

Examples of suitable excipients for the preparation of excipient based hydrous granules, includes, but are not limited to, microcrystalline cellulose, silicified microcrystalline cellulose, hydroxypropyl cellulose, hydroxypropyl methyl cellulose, lactose, sucrose, mannitol, starch, pregelatinized starch or other starch derivatives, magnesium aluminum silicate, magnesium aluminum metasilicate or any combinations thereof and co-processed excipients thereof.

The excipient based hydrous granule may be a microcrystalline cellulose based hydrous granule.

In another embodiment, the pharmaceutical composition of the present invention is a tablet. In one or more of the embodiments, the tablet of the present disclosure has a moisture content not more than 17% w/w, preferably between about 8-15% w/w.

The tablet comprises of a compressed core and a film coating over the compressed core.

The compressed core comprises a polyallylamine polymer and excipient based hydrous granules and has moisture content not more than 17% w/w, preferably between 7-14% w/w, more preferably between 9-13% w/w and most preferably around about 12% w/w.

In another embodiment, the tablet contains a moisture retaining agent. Suitable examples of moisture retaining agents includes but are not limited to silicon dioxide. The moisture retaining agent may be present in an amount ranging from about 0.5% to about 3% by weight, more preferably ranging from about 1% to about 2% by weight, most preferably ranging from about 1.25% to about 1.75% by weight of the composition.

In one or more embodiments, compressed core further comprises of a diluent, lubricant, binder, disintegrant, surfactant or combinations thereof.

Suitable examples of the lubricant include but are not limited to zinc stearate, talc, magnesium stearate, aluminum stearate, calcium stearate, polyethylene glycol, silica, colloidal silica, magnesium trisilicate, starches, tribasic calcium phosphate, stearic acid, sodium stearyl fumarate, magnesium carbonate, magnesium oxide, polyethylene glycol, glyceryl behenate, powdered cellulose, microcrystalline cellulose or combinations thereof. The lubricant(s) may be present in an amount ranging from about 0.1% to about 4% by weight of the composition.

Suitable examples of the disintegrant include, but are not limited to, starches, pregelatinized starches, alginates, low-substituted hydroxypropylcellulose, croscarmellose, crospovidone, sodium starch glycolate or combinations thereof. The disintegrant(s) may be present in an amount ranging from about 0.1% to about 12% by weight of the composition.

Suitable examples of the binder include, but are not limited to, hydroxyethyl cellulose, hydroxypropyl methylcellulose, cellulose derivatives, acacia, dextrin, carbomer, starch, povidone, carboxymethylcellulose, ethylcellulose, gelatin, maltose, guar gum, glucose, dextrin, methylcellulose, polymethacrylates, maltodextrin or combinations thereof. The binder(s) may be present in an amount ranging from about 1% to about 18% by weight of the composition.

Suitable examples of the diluents include, but are not limited to, microcrystalline cellulose, calcium carbonate, calcium phosphate, calcium sulfate, dextran, dextrose, fructose, kaolin, mannitol, anhydrous lactose, lactose monohydrate, maltose, sorbitol, sucrose, starch, pregelatinized starch, or combinations thereof. The diluent(s) may be present in an amount ranging from about 1% to about 30% by weight of the composition.

Suitable examples of the surfactant include, but are not limited to, sorbitan derivatives (such as Tween™, Span™), mono-, di- and polyglycerides, sugar derivatives (sucrose mono- and distearates), polyethylene glycol esters and ethers, polyethylene and polypropylene glycol block copolymers (such as Pluronic™, Poloxamer™), polyethoxylated oils (such as Cremophor™), sodium lauryl sulfate, and the like, and combinations thereof. The surfactant(s) may be present in an amount ranging from about 0% to about 2.5% by weight of the composition.

In one or more embodiments, the tablet comprises of a polymer based film coating. The Film coating may comprise of one or more of the cellulosic polymer like hydroxypropyl methyl cellulose (HPMC), hydrophilic agent like polyethylene glycol (PEG), Opacifier(s) like titanium dioxide (TiO₂), glident like talc or combinations thereof. Opacifiers suitable for use in the present invention, include, but are not limited to titanium dioxide, iron oxides, and the like, and combinations thereof. The Opacifiers may be present in an amount ranging from about 0.1 to about 1% by weight of the composition. The film coating may be present in an amount ranging from about 1% to about 5% by weight of the composition. The components of the film coat may be dissolved or dispersed in an appropriate solvent and the dispersion may be coated on the compressed core in a suitable coating equipment (such as a pan coater).

The pharmaceutical compositions as described herein may also contain permitted FD&C dyes and colors.

The Tablet prepared has an acceptable hardness, friability and disintegration time.

In some embodiments, the tablet has a hardness of between 14 to 22 kP, more preferably between 16 to 20 kP and most preferably around about 18 kP.

In one or more embodiments, the compressed core shows friability of less than 2%, more preferably of less than 1%.

The tablets demonstrate the tablet disintegration time of not more than 30 minutes, more preferably of not more than 20 minutes, even more preferably of not more than 10 minutes and most preferably of not more than 5 minutes.

Another aspect of the present disclosure relates to the processes for the preparation of the pharmaceutical compositions comprising polyallylamine polymer or salt thereof.

In one or more embodiments, the present disclosure provides processes for the preparation of the pharmaceutical compositions comprising of polyallylamine polymer or salts thereof, wherein the process does not involves the drying step. In preferred embodiments the pharmaceutical compositions prepared using the processes of the present invention is a tablet.

In one or more embodiments, the process for the preparation of the pharmaceutical compositions is a modified moisture activated granulation technique.

In one or more preferred embodiments, the present invention provides modified moisture activated granulation process for the preparation of tablet comprising of compressed core comprising of polyallylamine polymer or salt thereof and one or more pharmaceutically acceptable excipients.

In one or more embodiments, modified moisture activated granulation technique of the present invention comprises of preparation of compressed core by contacting the polyallylamine polymer with the excipient based hydrous granules to prepare mixture which can be further compressed. In preferred embodiments the modified moisture activated granulation technique comprises of activation of the polyallylamine polymer for compression by contacting it with excipient based hydrous granules. The resulting mixture can be further compressed.

In one or more embodiments, the modified moisture activated granulation technique includes addition of moisture retaining agent into the mixture of polyallylamine polymer and excipient based hydrous granules which can be further compressed to prepare a compressed core. Suitable examples of moisture retaining agent, include, but are not limited to, silicon dioxide. The moisture retaining agent may be present in an amount ranging from about 0.5% to 10% by weight of the composition, more preferably ranging from about 1% to 5% by weight of the composition.

In one or more embodiments, modified moisture activated granulation technique comprises of addition of suitable diluent, lubricant, binder, disintegrant, surfactant or combination thereof into the mixture of polyallylamine polymer, excipient based hydrous granules and optionally a moisture retaining agent. Mixture thus prepared can be compressed to prepare the compressed core.

Processes for the preparation of the pharmaceutical compositions according to the present invention comprises of various processes which include, but are not limited to, sifting, granulation, modified moisture activated granulation, lubrication, compression, coating and imprinting.

The pharmaceutical compositions as described herein may be prepared by process of modified moisture activated granulation technique. For example, polyallylamine polymer may be mixed with the excipient based hydrous granules and then with other optional ingredients such as moisture retaining agent(s), diluent, lubricant, binder, disintegrant, surfactant or combination thereof. Mixture may be then compressed by a tabletting machine, resulting in a compressed core.

The Film coat may be applied over the prepared compressed core. The components of the film coating may be dissolved or dispersed in the suitable solvent and then sprayed over the compressed core.

In one of the embodiments, the pharmaceutical composition comprises of a polyallylamine polymer that is prepared by first mixing polyallylamine with excipient based hydrous granules. The excipient based hydrous granules can be prepared by mixing microcrystalline cellulose and water to attain a moisture content of around about 23% by weight of the hydrous granules. Then, the final blend for compression can be prepared by optionally adding one or more of the moisture retaining agent(s), diluent, lubricant, binder, disintegrant, surfactant to the mixture of the polyallylamine polymer and excipient based hydrous granules. Followed by preparing the compressed core(s), by compressing thus prepared final blend for compression by using a conventional tabletting machine. Then, a film coating may be applied to the compressed core by spraying the film coating solution or suspension over the compressed core using suitable equipment such as a pan coater. Solution or suspension for film coating may be prepared by dissolving or dispersing the as herein described suitable components for film coating.

The present invention may further be illustrated by the following examples, which are not to be construed as limiting the invention.

### EXAMPLES

### Example 1

Example 1, as herein described, provides the procedure for the preparation of tablet using modified moisture activated granulation process according to the present invention. Table 1 as shown below shows the general ingredients used for the formulation of the tablet. As per the experiment conducted, table 2 shows the exemplary ingredients correlating to Table 1. Table 3 shows the results obtained after conducting the experiment employing the concentration of ingredients in Table 2.

In broad aspects, Tablet 1 gives ingredients for the preparation of tablet.

TABLE 1

| | | |
|---|---|---|
| S/N | Ingredient | % w/w |

Compressed core

| | | |
|---|---|---|
| 1 | Polyallylamine polymer | 50-95 |
| 2 | Excipient for hydrous granules | 5-20 |
| 3 | Purified Water | 6-12 |
| 4 | Moisture retaining agent | 0-3 |
| 5 | Lubricant | 0-2 |

Film coating

| | | |
|---|---|---|
| 6 | Polymer based film coating | 1-5 |
| 7 | Purified water | Q.s. |
| | Total | 100 |

TABLE 2

| | | | |
|---|---|---|---|
| S/N | Ingredient | mg/Tablet | % w/w |

Compressed core

| | | | |
|---|---|---|---|
| 1 | Sevelamer carbonate | 800.00** | 73.273 |
| 2 | Microcrystalline cellulose (MCC) | 130.00 | 11.907 |
| 3 | Purified Water | Q.s. | 9.526 |
| 4 | Silicon dioxide (Syloid 244 FP) | 18.00 | 1.648 |
| 5 | Zinc stearate (Synpro) | 8.00 | 0.733 |

Film coating

| | | | |
|---|---|---|---|
| 6 | Opadry Clear* | 31.80 | 2.913 |
| 7 | Purified water | Q.s | — |
| | Total | 1091.80 | 100.000 |

**863.5 mg of Sevelamer\carbonate (having about 7% w/w moisture content) equivalent to 800 mg Sevelamer carbonate on dry basis.

Silicon dioxide as used herein acts as a moisture retaining agent and helps to maintain moisture content and to prevent any possible loss of moisture content during the processing. Usage of silicon dioxide as moisture retaining agent is optional. In the case wherein silicon dioxide is not used as per the example 1, the amount of it may be compensated by MCC.

### Procedure

Powdered Microcrystalline Cellulosed is shifted through 40 mesh size and loaded into a Rapid Mixer Granulator (RMG). Sufficient amount of purified water is added as a binder solution for granulation of MCC followed by kneading to achieve proper microcrystalline cellulose based hydrous granules that have a moisture content about 23% by weight of the hydrous granules. Powdered Sevelamer Carbonate having moisture content around 7% w/w is shifted through 40 mesh and added in the RMG to microcrystalline cellulose based hydrous granules. Mixing of Sevelamer Carbonate with microcrystalline cellulose based hydrous granules in RMG is carried out to achieve proper granules. Followed by the entire granule mixture being sifted using 20 mesh. Extra granular component Syloid 244FP is passed through 40 mesh first and then is added into the prepared entire granule mixture. Zinc stearate is first passed through 60 mesh and into the prepared entire granule mixture and lubricated for about 3 minutes. Mixture thus prepared is ready for compression which has a loss on drying value of about 12% w/w. Mixture is then compressed to prepare compressed core. All the ingredients are taken in an amount as per their respective weight as described in the table above. Compressed cores are then taken into a pan coater device to apply coating solution Opadry clear with the weight gain of about 3.0% by weight of the composition. Optionally, the surface of the film coated tablet can be imprinted by using suitable imprinting ink such as opacode black in isopropanol.

### Characterization of Tablet of Example 1

Tablets prepared according to the present disclosure has the acceptable friability, hardness and disintegration time as shown in table 3.

TABLE 3

| Test Parameter | Result | Specification |
|---|---|---|
| Friability (compressed core i.e. | 0.05 | NMT 1%; |
| without film coating) | % w/w | Preferably NMT 0.3% |
| Hardness (compressed core i.e. | 19.09 kP | 14 to 22 kP |
| without film coating) | | |
| Disintegration time in 0.1N HCL | 3 min 25 | NMT 30 min |
| (film coated) | sec | Preferably NMT |
| | | 20 min; More |
| | | preferably NMT 5 |
| | | minutes |

### Comparative Example 1: Direct Compression

Table 4 as shown below depicts the ingredients used in the specified concentration in Direct Compression Technique. The results of which are shown further below in Table 5.

Dry mix of Sevelamer carbonate and MCC 112 is sifted through 40 mesh and then loaded into a V cone blender and mixed for 5 min at 25 rpm. Zinc stearate (passed

TABLE 4

| | | | |
|---|---|---|---|
| S/N | Ingredient | mg/Tablet | % w/w |

Dry mixture for direct compression

| | | | |
|---|---|---|---|
| 1 | Sevelamer carbonate | 800.0 | 76.90 |
| 2 | Microcrystalline cellulose | 200.0 | 19.22 |
| | (MCC Flocel 112) | | |
| 3 | Zinc stearate | 10.0 | 0.96 |

Film coating

| | | | |
|---|---|---|---|
| 4 | Opadry clear 06A590008 | 30.3 | 2.91 |
| 5 | Purified water | Q. S. | — |
| | Total | 1040.3 | 100.00 |

through 60#) is added into this mixture and blended for 3 min at 25 rpm. Followed by compressing the resultant mixture to prepare a tablet. Compressed cores are then taken into a pan coater device to apply coating solution Opadry clear with the weight gain of about 3.0% by weight of the composition.

### Characterization of Tablet of Comparative Example 1

TABLE 5

| Test Parameter | Result | Specification |
|---|---|---|
| Friability (compressed core i.e. | 0.63% | NMT 1%; |
| without film coating) | | Preferably NMT |
| | | 0.3% |
| Hardness (compressed core i.e. | 7-8 kP | 14 to 22 kP |
| without film coating) | | |
| Disintegration time in 0.1N HCL | 9 minutes 30 | NMT 20 min; |
| (film coated) | seconds | Preferably NMT |
| | | 5 minutes |

Tablets of polyallylamine polymer prepared by the direct compression are difficult to compress and have an unacceptable friability and hardness.

### Comparative Example 2: Wet Granulation

Table 6 as shown below depicts the ingredients used in the specified concentration in Wet Granulation Technique. The results of which are shown further below in Table 7.

TABLE 6

| | | | |
|---|---|---|---|
| S/N | Ingredients | mg/tablet | % w/w |

Wet granulation

| | | | |
|---|---|---|---|
| 1 | Sevelamer carbonate | 800.00 | 76.92 |
| 2 | Microcrystalline cellulose | 200.00 | 18.27 |
| | (Avicel PH 101) | | |
| 3 | Purified water | Q.s | — |
| 4 | Zinc stearate | 10 | 0.96 |

Film coating

| | | | |
|---|---|---|---|
| 5 | Opadry clear 06A590008 | 30.3 | 2.91 |
| 6 | Purified water | Q.s | — |

| | | |
|---|---|---|
| Total | 1040.30 | 100 |

### Procedure

Dry mix material Sevelamer and MCC Flocel 101 through 40 mesh is sifted. Sifted materials are loaded into a High shear granulator and dry mixed for 5 min followed by granulating the mixture with sufficient amount of water necessary for conventional wet granulation. Wet mass is dried at 60% inlet temperature and tried in order to achieve the loss on drying value of around 11-12%. Zinc stearate (passed through 60#) is added to the dried mass using a V cone blender and blended for about 3 min at 25 rpm. Compressed cores are prepared by compressing the mixture to their required weight as per the table above. Compressed cores are then taken into a pan coater device to apply coating solution Opadry clear with the weight gain of about 3.0% by weight of the composition.

### Characterization of Tablet Comparative Example 2

TABLE 7

| Test Parameter | Result | Specification |
|---|---|---|
| Friability (compressed core i.e. | 0.11% | NMT 1%; |
| without film coating) | | Preferably |
| | | NMT 0.3% |
| Hardness (compressed core i.e. | 8-9 kP | 14 to 22 kP |
| without film coating) | | |
| Disintegration time in 0.1N HCL | 5 minutes | NMT 20 min; |
| (film coated) | | Preferably |
| | | NMT 5 |
| | | minutes |

The tablets of polyallylamine polymer prepared by the conventional wet granulation process are difficult to compress and an acceptable hardness and friability is not achieved. Conventional wet granulation process, involves a drying step at 60° C. In this step it becomes difficult to achieve LOD in the desired range. Even If the material needs to be semi dried, it is still very difficult to control the desired range of LOD. Furthermore, in the wet granulation process, swelling of Sevelamer is greater as compared to the process applied in the present disclosure i.e. modified moisture activated granulation process. The swelled particles of polyallylamine polymers eventually shrink during drying. Compatibility of this re-shrinked material has been found to be unpredictable as well as raises the hurdle for the robust process for the preparation of the tablet comprising of polyallylamine polymer.

1. A pharmaceutical composition comprising a compressed core comprising a polyallylamine polymer as an active pharmaceutical ingredient (API) and at least one excipient based hydrous granule wherein said pharmaceutical composition has a moisture content ranging from about 6-17% w/w.

2. The pharmaceutical composition of claim 1, wherein hardness of said pharmaceutical composition is ranging from about 14 to 22 kP.

3. The pharmaceutical composition of claim 1, wherein friability of said pharmaceutical composition is not more than about 1%.

4. The pharmaceutical composition of claim 1, wherein disintegration time of said pharmaceutical composition is not more than about 30 minutes.

5. The pharmaceutical composition of claim 1, wherein said moisture content is ranging from about 8-15% w/w.

6. The pharmaceutical composition of claim 1, wherein said polyallylamine polymer is selected from the group consisting of sevelamer, colesevelam and pharmaceutically acceptable salts thereof.

7. The pharmaceutical composition of claim 1, wherein said polyallylamine polymer is sevelamer carbonate.

8. The pharmaceutical composition of claim 7, wherein sevelamer carbonate is present in an amount ranging from about 60% to about 85% by weight of the pharmaceutical composition.

9. The pharmaceutical composition of claim 7, wherein sevelamer carbonate is having a moisture content ranging from about 5-9% w/w.

10. The pharmaceutical composition of claim 1, wherein the at least one excipient based hydrous granule is a microcrystalline cellulose based hydrous granule.

11. The pharmaceutical composition of claim 1, wherein at least one excipient based hydrous granule is present in an amount ranging from about 10% to about 15% by weight of the pharmaceutical composition.

12. The pharmaceutical composition of claim 1, wherein the at least one excipient based hydrous granule has a moisture content ranging from about 10% w/w to 35% w/w.

13. The pharmaceutical composition of claim 1 further comprising at least one moisture retaining agent.

14. The pharmaceutical composition of claim 13, wherein the at least one moisture retaining agent is silicon dioxide.

15. The pharmaceutical composition of claim 13, wherein the at least one moisture retaining agent is present in amount ranging from about 0.5% to about 3% by weight of the pharmaceutical composition.

16. The pharmaceutical composition of claim 13, further comprises an additive selected from the group consisting of a diluent, a lubricant, a binder, a disintegrant, a surfactant and a combination thereof.

17.-18. (canceled)

19. The pharmaceutical composition of claim 16, wherein composition comprises:
a. colesevelam or sevelamer or pharmaceutically acceptable salts thereof in an amount about 50% to about 95% by weight of the composition;
b. microcrystalline cellulose based hydrous granules in an amount about 5% to about 30% by weight of the composition;
c. a moisture retaining agent preferably silicon dioxide in an amount about 0.5% to about 3% by weight of the composition;
d. a lubricant preferably zinc stearate in an amount about 0.1% to about 2% by weight of the composition; and
e. optionally polymer based film coating.

20. A modified moisture activated granulation process for preparation of tablet comprising steps of:
a. preparing excipient based hydrous granules;
b. contacting or mixing polyallylamine polymer with the excipient based hydrous granules of step a;
c. optionally adding a moisture retaining agent to the mixture product obtained from step b;
d. optionally adding a lubricating agent to the mixture obtained from the step b or step c;
e. preparing a compressed core by compressing the product obtained from the step b, step c or step d;
f. optionally applying a film coat over the compressed core obtained in step e; wherein the moisture content of the product of step f is between about 7-17% w/w.

21. The process according to claim 20, wherein, said polyallylamine polymer is sevelamer carbonate.

22. The process according to claim 18, wherein, the excipient based hydrous granules are microcrystalline cellulose based hydrous granules.

23. (canceled)
